(19) United States
(12) Patent Application Publication
(10) Pub. No.: US 2016/0350138 A1
(43) Pub. Date: Dec. 1, 2016

(54) BIOFEEDBACK SYSTEM

(71) Applicant: Roya Caroline SALAS, Chestnut Hill, MA (US)

(72) Inventor: Roya Caroline SALAS, Chestnut Hill, MA (US)

(21) Appl. No.: 15/169,130

(22) Filed: May 31, 2016

Related U.S. Application Data
(60) Provisional application No. 62/168,890, filed on May 31, 2015.

(51) Int. Cl. G06F 9/44; G06F 3/01; A61B 5/00; G06T 13/80

(52) U.S. Cl. CPC G06F 9/4446; G06T 13/80; G06F 3/015; A61B 5/486

(57) ABSTRACT

A virtual biofeedback system is provided. The virtual biofeedback system provides visualization animation related to parts of the human body subject to one or more predetermined conditions, wherein the visualization animations are adapted to assist an instructed user to visualize an improved, optimal functioning of the subject body part.

### CROSS-REFERENCE TO RELATED APPLICATION

This application claims the benefit of priority of U.S. provisional application No. 62/168,890, filed 31 May 2015, the contents of which are herein incorporated by reference.

### BACKGROUND OF THE INVENTION

The present invention relates to therapeutic systems and, more particularly, an online biofeedback system providing visualization animation related to parts of the human body subject to one or more predetermined conditions, wherein the visualization animations are adapted to assist an instructed user to visualize an improved, optimal functioning of the subject body part.

There is evidence that your attitude toward life can improve your health and even speed your recovery from a serious ailment or surgery. The attitudes that seem to help the most are optimism, hope, and a feeling that you have some impact on the quality of your own life.

The applicant has discovered that one can feel that they have some limited control over their predetermined condition by visualizing the internal workings of their bodies and looking at images that show their predetermined condition improving, be that predetermined condition a headache, cancer and everything in between. Similarly, one's mind can influence one's immune system, for example, by visualizing the disease condition improving, the user can have a positive influence on his or her health. Furthermore, visualization with specificity—i.e., the specific problem and the specific goal—tends to improve the performance and effects of any therapeutic regime. The specificity of the medical problem may be determined through biofeedback, with it being understood that feedback is the basis for improving any system the feedback loop is engaged with.

Current biofeedback machines require that the user be “plugged” into them or otherwise require a physical connection. For example, existing biofeedback machines for alerting users to their stress level are connected to the user to determine skin and/or finger temperature to do so, but they are not very helpful in improving the stress level and/or heart rate of the user. This required-physicality limits current biofeedback machines' portability, affordability and ease of use, and, again, they do not actively help users improved their identified conditions. Furthermore, if a user has multiple conditions, it would require multiple biofeedback machines that they would need to physically interface with.

As can be seen, there is a need for an online biofeedback system providing visualization animation related to parts of the human body subject to one or more predetermined conditions, wherein the visualization animations are adapted to assist an instructed user to visualize an improved, optimal functioning of the subject body part.

### SUMMARY OF THE INVENTION

In one aspect of the present invention, a computer-implemented biofeedback system includes a display device; a user interface; a database configured to store and selectively retrieve a plurality of visualization animation representative of human body parts functioning optimally and functioning subject to a plurality of predetermined conditions; a computer operatively connected to the display device, database and the user interface, wherein the computer is configured to prompt a user through the user interface to identify one or more predetermined conditions, wherein the computer is configured to associate each identified predetermined condition with at least one human body part, and wherein the computer is configured to selectively retrieve from the database at least two visualization animations representative of the at least one human body part functioning optimally and functioning subject to each identified predetermined conditions; and the database configured to display through the display device said at least two visualization animations associated with each identified predetermined conditions.

In another aspect of the present invention, the computer-implemented biofeedback system the computer is further configured to transmit through the user interface or the display device a set of instructions advising the user how to engage said at least two visualization animations, wherein the set of instructions defines a sequential order for viewing said at least two visualization animations, wherein the set of instructions prompts the user to envision improvement of each identified predetermined condition when viewing said at least two visualization animations, wherein the set of instructions prompts the user to envision how the one or more associated human body parts would look if each identified predetermined condition were non-existent, further including at least one external biofeedback device operatively connected to the computer, wherein identification of each identified predetermined condition is based in part on output of the at least external biofeedback device.

In another aspect of the present invention, a computer-implemented method of providing virtual biofeedback includes prompting a user to identify one or more predetermined conditions; associating each prompted predetermined condition with at least one human body parts; and displaying through a display device at least two visualization animations representative of each associated human body part functioning optimally and functioning subject to the one or more predetermined conditions, further providing a set of instructions advising the user how to engage said at least two visualization animations, wherein the set of instructions defines a sequential order for viewing said at least two visualization animations, wherein the set of instructions prompts the user to envision improvement of each identified predetermined condition when viewing said at least two visualization animations, and wherein the set of instructions prompts the user to envision how the one or more associated human body parts would look if each identified predetermined condition were non-existent.

These and other features, aspects and advantages of the present invention will become better understood with reference to the following drawings, description and claims.

### BRIEF DESCRIPTION OF THE DRAWINGS

FIG. 1 is a schematic view of an exemplary embodiment of the present invention;

FIG. 2 is a schematic view of an exemplary embodiment of the present invention;

FIG. 3 is a schematic view of an exemplary embodiment of a website of the present invention; and

FIG. 4 is a schematic view of an exemplary embodiment of a system server of the present invention.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Broadly, an embodiment of the present invention provides an online biofeedback system providing visualization animation related to parts of the human body subject to one or more predetermined conditions, wherein the visualization animations are adapted to assist an instructed user to visualize an improved, optimal functioning of the subject body part.

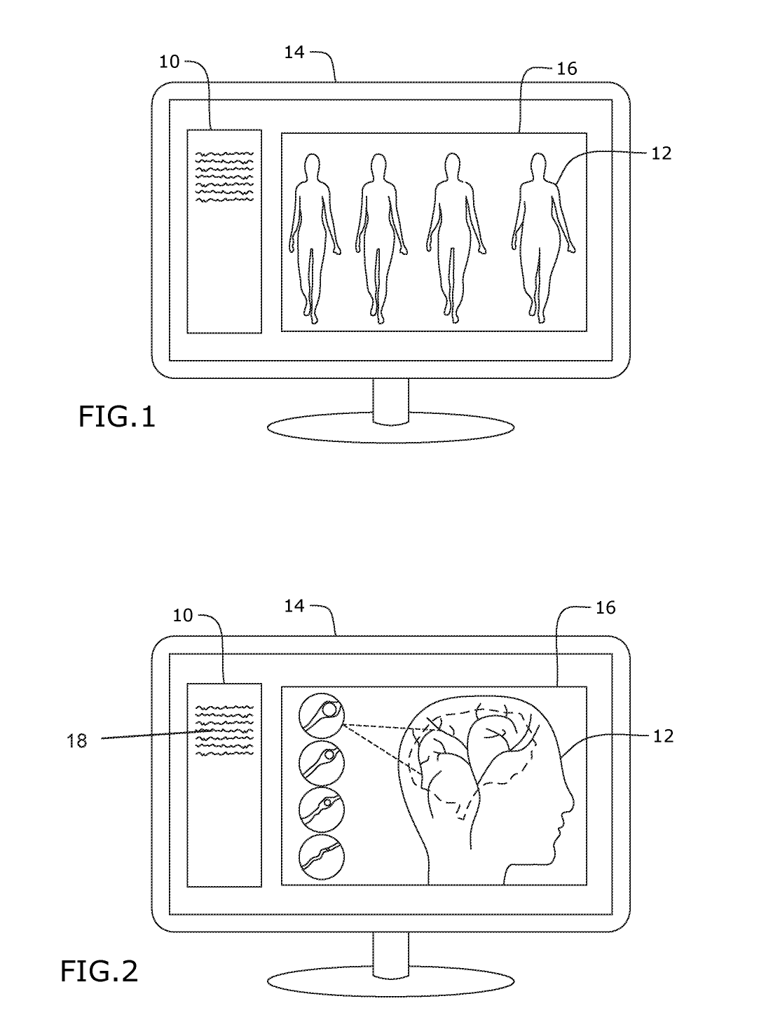

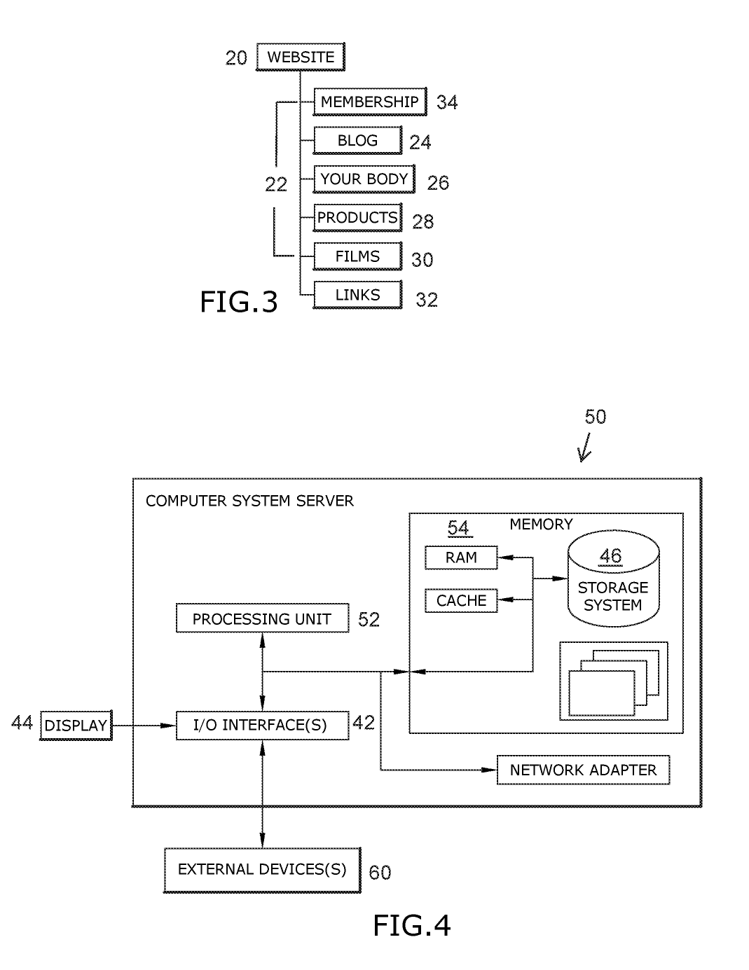

Referring now to FIGS. 1 through 4, the present invention may include at least one computer 50 with a user interface. The computer 50 may include any processor 52 unit having a form of memory 54 including, but not limited to, a desktop, laptop, and smart device, such as, a tablet and smart phone. The computer 50 includes a program product including a machine-readable program code for causing, when executed, the computer 50 to perform steps. The program product may include software which may either be loaded onto the computer 50 or accessed by the computer 50. The loaded software may include an application on the computer 50/smart device. The software may be accessed by the computer 50 using a web browser. The computer 50 may access the software via the web browser using the internet, extranet, intranet, host server, internet cloud, communication channel, wireless communication channel, and the like. The computer 50 may be operatively connected to a database 46.

The present invention may provide a virtual site 20 accessibly through a communication channel. The virtual site 20 may be, but not limited to, an online website or database 46 accessible through the Internet. The virtual site 20 may have a membership functionality 34 for secure enrollment and describing different ways a user can navigate the virtual site 20. The virtual site 20 may provide biofeedback content 22 enabling users to receive detailed information about the internal workings of their body. The biofeedback content 22 may be conveyed through written text 10 and visualization animation 12. Visualization animation 12 may include words, imagery, animation, audio content and the like adapted to convey a better understand of how the user's body works optimally, how a plurality of predetermined conditions affects the user's body, and what needs to happen in their body to make each predetermined condition better.

In an exemplary embodiment, the system may be utilized by an individual who is suffering from a headache. A headache is often the result of dilated blood vessels. The visualization animation 12 on the system can help the user visualize their head and concentrate on the area where they are feeling pain. As illustrated in FIG. 2, the visualization animation 12 can then show the dilation of the blood vessels decreasing and the vessels going back to normal. The user is encouraged to visualize the same thing happening to them, helping them to get rid of their headache.

In certain embodiments, the biofeedback content 22 provides a set of instructions 18 on how to identify a plurality of conditions, and the set of instructions 18 instruct users on how to focus on improving the identified condition(s). The set of instructions 18 may be conveyed through the written text 10, through audio or visual data, or the like. Specifically, the system may provide visual representations 16 (visualization animation 12), for example, over the user interface 42 and/or connected display monitor 14, of the parts of the body affected by the identified condition(s), wherein such visualization animation 12 represent the affected body parts functioning subject to the predetermined condition(s) and functioning optimally. The system of the present invention then, through the set of instructions 18, may prompt the user to (a) focus in their mind visualizations of their own “system” (affected body parts) behaving akin to the optimal functionality, and (b) visualize their condition improving accordingly.

Additionally, in an exemplary embodiment, the user is shown visualization animation 12 including images of cancer cells and animation showing the cancer cells being destroyed or reducing in size, helping the user visualize the same thing happening to them.

Additionally, in an exemplary embodiment, the user is shown visualization animation 12 including relaxing images of their choosing accompanied with music to help them with stress relief and encourage a sense of wellbeing.

Additionally, in an exemplary embodiment, the user who has arthritis can look at visualization animation 12 showing reduced inflammation in the joints and increased movement to help improve their condition.

In another exemplary embodiment, the system may be used for individuals to motivate themselves to exercise or eat healthier by watching visualization animation 12 of them doing these things.

The present invention, in certain embodiments through the user interface 42, may provide links 32 to the biofeedback content 22. In certain embodiments, the visualization animation 12 and/or the biofeedback content 22 may be embodied in films or other visual media 30, products 28, and/or blogs 24. The biofeedback content 22 may be categorized by searchable topics 26, such as body parts, predetermined conditions, improved states of being, and the like.

The present invention does not require the user to be physically connected to the system, nor is the system only applicable to a limited use or body part or condition or set of conditions. Rather, the present invention allows, through the website and or app, the user to pick what predetermined condition they want addressed and click on it. As a result, they would be prompted by the set of instructions 18 to interact with the visual representations 16 of related visualization animation 12 adapted to improve their predetermined condition(s).

The present invention may be adapted for receiving input and output from pre-existing biofeedback machines 60; for example the above-mentioned stress testing machine may be electronically or electrically connected to the computer 50 50/database 46 of the present invention so that the computer 50 50/database 46 may utilize the data collected by the biofeedback machine 60 to identify one or more predetermined condition.

The present invention will show images and short movies to help train a user's subconscious to assist them in obtaining optimal health and achieving your goals.

The computer 50-based data processing system and method described above is for purposes of example only, and may be implemented in any type of computer 50 system or programming or processing environment, or in a computer 50 program, alone or in conjunction with hardware. The present invention may also be implemented in software stored on a computer 50-readable medium and executed as a computer 50 program on a general purpose or special purpose computer 50. For clarity, only those aspects of the system germane to the invention are described, and product details well known in the art are omitted. For the same reason, the computer 50 hardware is not described in further detail. It should thus be understood that the invention is not limited to any specific computer 50 language, program, or computer 50. It is further contemplated that the present invention may be run on a stand-alone computer 50 system, or may be run from a server computer 50 system that can be accessed by a plurality of client computer 50 systems interconnected over an intranet network, or that is accessible to clients over the Internet. In addition, many embodiments of the present invention have application to a wide range of industries. To the extent the present application discloses a system, the method implemented by that system, as well as software stored on a computer 50-readable medium and executed as a computer 50 program to perform the method on a general purpose or special purpose computer 50, are within the scope of the present invention. Further, to the extent the present application discloses a method, a system of apparatuses configured to implement the method are within the scope of the present invention.

It should be understood, of course, that the foregoing relates to exemplary embodiments of the invention and that modifications may be made without departing from the spirit and scope of the present invention.

What is claimed is:

1. A computer-implemented biofeedback system, comprising:
a display device;
a user interface;
a database configured to store and selectively retrieve a plurality of visualization animation representative of human body parts functioning optimally and functioning subject to a plurality of predetermined conditions;
a computer operatively connected to the display device, database and the user interface, wherein the computer is configured to prompt a user through the user interface to identify one or more predetermined conditions, wherein the computer is configured to associate each identified predetermined condition with at least one human body part, and wherein the computer is configured to selectively retrieve from the database at least two visualization animations representative of the at least one human body part functioning optimally and functioning subject to each identified predetermined conditions; and
the database configured to display through the display device said at least two visualization animations associated with each identified predetermined conditions.

2. The computer-implemented biofeedback system of claim 1, wherein the computer is further configured to transmit through the user interface or the display device a set of instructions advising the user how to engage said at least two visualization animations.

3. The computer-implemented biofeedback system of claim 2, wherein the set of instructions defines a sequential order for viewing said at least two visualization animations.

4. The computer-implemented biofeedback system of claim 3, wherein the set of instructions prompts the user to envision improvement of each identified predetermined condition when viewing said at least two visualization animations.

5. The computer-implemented biofeedback system of claim 4, wherein the set of instructions prompts the user to envision how the one or more associated human body parts would look if each identified predetermined condition were non-existent.

6. The computer-implemented biofeedback system of claim 5, further comprising at least one external biofeedback device operatively connected to the computer.

7. The computer-implemented biofeedback system of claim 6, wherein identification of each identified predetermined condition is based in part on output of the at least external biofeedback device.

8. A computer-implemented method of providing virtual biofeedback, comprising:
prompting a user to identify one or more predetermined conditions;
associating each predetermined condition with at least one human body parts; and
displaying through a display device at least two visualization animations representative of each associated human body part functioning optimally and functioning subject to the one or more predetermined conditions.

9. The computer-implemented method of claim 8, further providing a set of instructions advising the user how to engage said at least two visualization animations.

10. The computer-implemented method of claim 9, wherein the set of instructions defines a sequential order for viewing said at least two visualization animations.

11. The computer-implemented method of claim 10, wherein the set of instructions prompts the user to envision improvement of each identified predetermined condition when viewing said at least two visualization animations.

12. The computer-implemented method of claim 11, wherein the set of instructions prompts the user to envision how the one or more associated human body parts would look if each identified predetermined condition were non-existent.
